(19) United States
(12) Patent Application Publication
(10) Pub. No.: US 2008/0057071 A1
(43) Pub. Date: Mar. 6, 2008

(54) Use Of Hedgehog Pathway Inhibitors In Small-Cell Lung Cancer

(76) Inventors: David N. Watkins, Baltimore, MD (US); David M. Berman, Towson, MD (US); Stephen B. Baylin, Baltimore, MD (US); Philip A. Beachy, Towson, MD (US)

Correspondence Address: DLA PIPER US LLP, 4365 EXECUTIVE DRIVE, SUITE 1100, SAN DIEGO, CA, 92121-2133, US

(21) Appl. No.: 10/576,149

(22) PCT Filed: Oct. 20, 2004
(86) PCT No.: PCT/US04/34534

§ 371 Date: Jan. 23, 2007

Related U.S. Application Data
(60) Provisional application No. 60/512,651, filed on Oct. 20, 2003.

(51) Int. Cl. A61K 39/395; A61K 31/42; A61P 35/00; C12N 5/08; C12Q 1/68; G01N 33/53

(52) U.S. Cl. 424/158.1; 435/375; 435/6; 435/7.8; 514/12; 514/278

(57) ABSTRACT

Elevated Hedgehog (Hh) pathway activity, including ligand stimulated Hh pathway activity, was detected in small-cell lung cancer (SCLQ cells, and determined to be associated with growth and proliferation of the cancer cells. Accordingly, methods are provided for treating SCLC associated with elevated Hh pathway activity by reducing or inhibiting the Hh pathway activity. Also provided are methods of determining the responsiveness of SCLC to treatment with a Hh pathway antagonist.

### CROSS REFERENCE TO RELATED APPLICATIONS

This application claims the benefit of priority under 35 U.S.C. § 119(e) of U.S. Ser. No. 60/512,651, filed Oct. 20, 2003, the entire content of which is incorporated herein by reference.

### BACKGROUND OF THE INVENTION

1. Field of the Invention

The present invention relates generally to the use of compounds to treat a variety of disorders, diseases and pathologic conditions and more specifically to the use of Hedgehog antagonists for inhibiting hedgehog pathway activity in small-cell lung cancer (SCLC).

2. Background Information

Pattern formation is the activity by which embryonic cells form ordered spatial arrangements of differentiated tissues. Speculation on the mechanisms underlying these patterning effects usually centers on the secretion of a signaling molecule that elicits an appropriate response from the tissues being patterned. More recent work aimed at the identification of such signaling molecules implicates secreted proteins encoded by individual members of a small number of gene families.

Members of the Hedgehog family of signaling molecules mediate many important short- and long-range patterning processes during invertebrate and vertebrate development. Exemplary hedgehog genes and proteins are described in PCT publications WO 95/18856 and WO 96/17924. The vertebrate family of hedgehog genes includes at least four members, three of which, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. Desert hedgehog (Dhh) is expressed principally in the testes, both in mouse embryonic development and in the adult rodent and human; Indian hedgehog (Ihh) is involved in bone development during embryogenesis and in bone formation in the adult; and, Shh is primarily involved in morphogenic and neuroinductive activities. Given the critical inductive roles of hedgehog polypeptides in the development and maintenance of vertebrate organs, the identification of hedgehog interacting proteins and their role in the regulation of gene families known to be involved in cell signaling and intercellular communication provides a possible mechanism of small-cell lung cancer (SCLC) suppression.

Without treatment, SCLC has the most aggressive clinical course of any type of pulmonary tumor, with median survival from diagnosis of only 2 to 4 months. Compared with other cell types of lung cancer, SCLC has a greater tendency to be widely disseminated by the time of diagnosis, but is much more responsive to chemotherapy and irradiation. Therefore the aim of early detection programs is to diagnose the cancer at an early curable stage.

The role of Hh pathway activity in promoting metastatic growth suggests that pathway antagonists may offer significant therapeutic improvements in the treatment of SCLC. The ability to modulate one or more genes that are part of the hedgehog signaling cascade thus represents a possible therapeutic approach to several clinically significant cancers. A need therefore exists for methods and compounds that inhibit signal transduction activity by modulating activation of a hedgehog, patched, or smoothened-mediated signal transduction pathway, such as the Hedgehog signaling pathway, to reverse or control aberrant growth related to SCLC.

### SUMMARY OF THE INVENTION

The present invention is based, in part, on the observation that Hedgehog (Hh) pathway activity is elevated in small-cell lung cancer (SCLC) cells as compared to corresponding normal cells of the organ with the tumor, and that agents that decrease the Hh pathway activity inhibit proliferation or metastasis of SCLC cells. Hh ligands that can stimulate Hh pathway activity include Sonic hedgehog (SHH), Indian hedgehog (IHH), and/or Desert hedgehog (DHH). Elevated Hh pathway activity also can be due, for example, to a mutation in a Hh ligand receptor such as Patched (PTCH), wherein PTCH in inactivated, resulting in unregulated Smoothened (SMO) activity and elevated Hh pathway activity. Accordingly, the present invention provides methods of treating SCLC characterized by elevated Hh pathway activity, as well as methods of determining whether a SCLC tumor has such activity and methods of identifying agents useful for treating such tumors. As such, methods of are provided wherein agents can be selected that are particularly useful for treating SCLC in a subject.

The present invention relates to a method of reducing or inhibiting proliferation or metastasis of SCLC cells characterized by elevated Hh pathway activity. Such a method can be performed, for example, by contacting the cells with at least one (e.g., 1, 2, 3, 4, or more) Hh pathway antagonist, whereby proliferation or metastasis of the SCLC cells is reduced or inhibited. The Hh pathway generally includes a Hh ligand (e.g., SHH, IHH and/or DHH), which binds a Hh ligand receptor (e.g., PTCH), resulting in activation of SMO (a G-protein coupled receptor-like polypeptide), which transduces the Hh signal downstream, resulting in activation of additional members of the Hh pathway (e.g., Fused), including Hh pathway stimulated transcription factors (e.g., members of the GLI family of transcription factors such as GLI-1). Also associated with Hh pathway activity are transcriptional targets, including, for example, nestin and BMP4, which can be induced by activated GLI transcription factor. As such, it will be recognized that a Hh pathway antagonist useful in a method of the invention is selected, in part, in that it acts at or downstream of the position in the Hh pathway associated with the elevated Hh pathway activity. For example, where elevated Hh pathway activity is ligand stimulated, the Hh antagonist can be selected based on the ability to sequester the Hh ligand or to reduce or inhibit binding of the Hh ligand to its receptor, or at any point downstream of these events. In comparison, where elevated Hh pathway activity is due to an inactivating mutation of the Hh ligand receptor (e.g., PTCH), the Hh pathway antagonist can be selected based on the ability, for example, to bind to and inhibit SMO or to reduce the activity of an activating GLI transcription factor (e.g., GLI-1 or GLI-2), but not at a point upstream.

Thus, in one embodiment, the invention provides a method of ameliorating SCLC in a subject. Such a method can be performed by administering to the subject at least one Hh pathway antagonist such that the Hh pathway antagonist contacts SCLC cells in the subject. According to the present method, the Hh pathway antagonist(s) can reduce or inhibit proliferation or metastasis of the tumor cells, thereby ameliorating the SCLC in the subject.

A SCLC tumor in a subject to be treated exhibits elevated Hh pathway activity (e.g., elevated ligand stimulated Hh pathway activity). Hh pathway antagonist(s) can be administered in any way typical of an agent used to treat the particular type of pulmonary tumor. For example, the Hh pathway antagonist(s) can be administered orally or parenterally, including, for example, by injection or inhalation, or by any combination of such methods.

The Hh pathway antagonist can be any type of compound as disclosed herein or otherwise having the ability to interfere with Hh pathway activity. In one embodiment, the Hh pathway antagonist is an antibody, for example, an antibody specific for one or more Hh ligand(s) (e.g., an anti-SHH, anti-IHH, and/or anti-DHH antibody). In another embodiment, the Hh pathway antagonist is a SMO antagonist such as a steroidal alkaloid, or a derivative thereof (e.g., cyclopamine, KAAD-cyclopamine, or jervine), or other synthetic small molecule such as SANT-1, SANT-2, SANT-3, or SANT-4. In still another embodiment, a combination of Hh pathway antagonists are administered to the subject. Further, any additional compounds that can provide a therapeutic benefit can be administered to the subject, including, for example, a chemotherapeutic agent or nutritional supplement, and/or the subject can be further treated, for example, by radiation therapy or using a surgical procedure.

The present invention further relates to a method of identifying SCLC of a subject amenable to treatment with a Hh pathway antagonist. As such, the method provides a means to determine whether a subject having SCLC is likely to be responsive to treatment with a Hh pathway antagonist. The method can be performed, for example, by detecting elevated Hh pathway activity in a sample of SCLC cells of the subject as compared to corresponding normal cells, wherein detection of an elevated level indicates that the subject can benefit from treatment with a Hh pathway antagonist. The sample of cells can be any sample, including, for example, a tumor sample obtained by biopsy of a subject having the tumor, a tumor sample obtained by surgery (e.g., a surgical procedure to remove and/or debulk the tumor), or a sample of the subject's bodily fluid (e.g., sputum or lung aspirate). The Hh pathway activity can be elevated due, for example, to a mutation of a gene encoding a Hh pathway polypeptide (e.g., an inactivating mutation of PTCH), or can be elevated due to ligand stimulated Hh pathway activity.

In one embodiment, the method of identifying SCLC amenable to treatment with a Hh pathway antagonist includes detecting an abnormal level of expression of one or more Hh pathway polypeptide(s), including, for example, one or more Hh ligands (e.g., SHH, IHH, and/or desert hedgehog), Hh ligand receptors (e.g., PTCH), or transcription factors (a GLI family member such as GLI-1). In one embodiment, the abnormal expression is an elevated expression of one or more Hh pathway polypeptide(s), including, for example, one or more Hh ligands (e.g., SHH, IHH, and/or desert hedgehog), Hh ligand receptors (e.g., PTCH), or transcription factors (a GLI family member such as GLI-1), or a combination of such Hh pathway polypeptides. In another embodiment, the abnormal level of expression is a reduced or suppressed expression of one or more Hh pathway polypeptide(s), including, for example, GLI-3, which acts as a transcriptional repressor in the Hh pathway. Increased or decreased expression of a Hh pathway polypeptide can be detected by measuring the level of a polynucleotide encoding the Hh pathway polypeptide using, for example, a hybridization assay, a primer extension assay, or a polymerase chain reaction assay (e.g., measuring the level of PTCH mRNA expression and/or GLI mRNA expression); or by measuring the level the Hh pathway polypeptide(s) using, for example, an immunoassay or receptor binding assay.

In another embodiment, the method of identifying SCLC amenable to treatment with a Hh pathway antagonist includes detecting an elevated activity of one or more Hh pathway polypeptide(s). For example, elevated activity of Hh pathway transcription factor (e.g., a GLI family member such as GLI-1) can be detected by measuring increased binding activity of the transcription factor to a cognate transcription factor regulatory element (e.g., using an electrophoretic mobility shift assay); by measuring increased expression of a reporter gene comprising a cognate transcription factor regulatory element; or measuring expression of GLI and/or of PTCH, and/or a target of the GLI transcription factor (e.g., by detecting transcription of nestin or BMP4). In still another embodiment, the method can include detecting expression of a Hh pathway polypeptide having an inactivating mutation, wherein the mutation is associated with elevated Hh pathway activity (e.g., by detecting expression of a mutant PTCH Hh ligand receptor).

The method of identifying SCLC amenable to treatment with a Hh pathway antagonist can further include contacting cells of the sample with at least one Hh pathway antagonist, and detecting a decrease in Hh pathway activity in the cells following said contact. The decreased Hh pathway activity can be detected, for example, by measuring decreased expression of a reporter gene regulated by a Hh pathway transcription factor, or by detecting a decrease in proliferation of the tumor cells. Such a method provides a means to confirm that the SCLC is amenable to treatment with a Hh pathway antagonist. Further, the method can include testing one or more different Hh pathway antagonists, either alone or in combination, thus providing a means to identify one or more Hh pathway antagonists useful for treating the particular SCLC being examined.

The present invention further relates to a method of identifying an agent useful for treating SCLC cells having elevated Hh pathway activity. In one embodiment, the method provides a means for practicing personalized medicine, wherein treatment is tailored to the particular patient based on the characteristics of the SCLC in the patient. The present method can be practiced, for example, by contacting a sample of SCLC cells with at least one test agent, wherein a decrease in Hh pathway activity in the presence of the test agent as compared to Hh pathway activity in the absence of the test agent identifies the agent as useful for treating SCLC. Also provided are methods for monitoring a therapeutic regimen for treating a subject having SCLC by determining a change in Hh pathway activity during therapy.

The present method can be practiced using test agents that are known to be effective in treating cancers having elevated Hh pathway activity in order to identify one or more agents that are particularly useful for treating the SCLC being examined, or using test agents that are being examined for effectiveness. As such, in one aspect, the test agent examined according to the present method can be any type of compound, including, for example, a peptide, a polynucleotide, a peptidomimetic, or a small organic molecule, and can be one of a plurality of similar but different agents (e.g., a combinatorial library of test agents, which can be a randomized or biased library or can be a variegated library based on known effective agents). In another aspect, the test agent comprises a known Hh pathway antagonist such as an antibody (e.g., an anti-SHH antibody and/or anti-IHH antibody), a steroidal alkaloid or a derivative thereof (e.g., cyclopamine, jervine, or triparanol), or a combination thereof.

Generally, though not necessarily, the method is performed by contacting the sample of cells ex vivo, for example, in a culture medium or on a solid support. As such, the methods are conveniently adaptable to a high throughput format, wherein a plurality (i.e., 2 or more) of samples of cells, which can be the same or different, are examined in parallel. Thus in one embodiment, test agents can be tested on several samples of cells from a single subject, allowing, for example, for the identification of a particularly effective concentration of an agent to be administered to the subject, or for the identification of a particularly effective agent to be administered to the subject. In another embodiment, a high throughput format allows for the examination of two, three, four, etc., different test agents, alone or in combination, on the SCLC cells of a subject such that the best (most effective) agent or combination of agents can be used for a therapeutic procedure. Accordingly, in various embodiments, the high throughput method is practiced by contacting different samples of cells of different subjects with same amounts of a test agent; or contacting different samples of cells of a single subject with different amounts of a test agent; or contacting different samples of cells of two or more different subjects with same or different amounts of different test agents. Further, a high throughput format allows, for example, control samples (positive controls and/or negative controls) to be run in parallel with test samples, including, for example, samples of cells known to be effectively treated with an agent being tested. Variations of the exemplified methods also are contemplated.

### BRIEF DESCRIPTION OF THE DRAWINGS

FIG. 1a is a pictoral diagram showing that immunohistochemical detection of Shh and GLI-1 in adult mouse airways is negative in normal airways (left panels), but positive for both Shh and GLI-1 in serial sections 3 days after naphthalene injury (middle panels). By 4 days after naphthalene treatment (right panels), GLI-1-positive cells are reduced in number (arrow). Serial sections demonstrate that nascent CGRP-positive cells do not express stained GLI-1. Scale bar, 50 μm.

FIG. 1b is a graphical representation showing quantitative analysis of the bronchial epithelial staining in 1a (n=4, mean±s.e.m.).

FIG. 1c is a pictoral diagram showing Shh signaling in E13.5 lungs. Shh imununostaining in embryonic airway epithelium is shown in the left panel. The right panel shows X-gal staining of lungs obtained from E13.5 Ptch-LacZ mouse embryos, demonstrating intense mesenchymal staining. Scale bar, 25 μm.

FIG. 1d is a pictoral diagram showing clusters of LacZ-positive cells (arrows) in the airway epithelium of E16.5 (left panel) and adult (right panel) mice. Scale bar, 25 μm. a, airway; m, mesenchyme; bm, basement membrane.

FIG. 1e is a pictoral diagram showing confocal immunofluorescence detection of Hh signaling in lung development. The top row demonstrates expression of both CGRP and Ptch in an E16.5 airway (arrow), similar to that shown in 1d. The bottom row shows expression of CGRP (arrow) adjacent to Shh-expressing epithelial cells (see high-magnification inset). Scale bar, 25 μm.

FIG. 2a is a pictoral diagram showing examples of Shh and GLI-1 immunostaining in human lung cancer tissue. Note the widespread co-expression of Shh and GLI-1 in SCLC, which is reduced in the NSCLC example.

FIG. 2b is a pictoral diagram showing expression of Hh signaling components in lung cancer cell lines. The top panel shows immunoblotting (IB) data for expression of Shh, GUI and GAPDH. The bottom panel demonstrates Ptch mRNA expression in the same cell lines detected by RNAse protection assay (RPA). The markers along the right indicate relative molecular mass.

FIG. 2c is a graphical representation showing induction of Gli-luciferase activity in Shh-LIGHT2 reporter cells co-cultured with purified Shh-Np or the cell lines indicated on the x axis. Luciferase activity is normalized to a Renilla luciferase internal control (n=6, mean±s.e.m.).

FIG. 2d is a pictoral diagram showing Shh and GLI-1 expression in NCI-H249 SCLC xenograft cells detected by dual-label immunohistochemistry. The left panel shows a tumor cell expressing Shh alone (arrow); the right panel shows a Shh-expressing tumor cell (top arrow) and an adjacent GUI-expressing tumor cell (bottom arrow).

FIG. 3a is a graphical representation showing growth of cancer cell lines treated with monoclonal antibodies against β-galactosidase (β-gal) as a control, or Shh for 4 days.

FIG. 3b is a graphical representation showing NCI-H249 SCLC cell growth after 5 days, treated with tomatidine, cyclopamine or KAAD cyclopamine at the indicated concentrations.

FIG. 3c is a graphical representation showing the identical experiment of 3b performed in NCI-H157 NSCLC cells.

FIG. 3d is a graphical representation showing the response of stably transfected NCI-H249 SCLC cells to treatment with cyclopainine when expressing neomycin resistance (Neo^{r}, a mutant GLI-1 lacking the zinc finger domain (Flag-Gli1ZFD), GLI-1 (Flag-Gli1), and wild-type untransfected (WT) cells. Cell viability was measured by MTT assay, detected at an absorbance at 540 nm (A₅₄₀) (n=6) and expressed as a percentage of control±s.e.m.

FIG. 3e is a pictoral diagram showing cell cycle analysis in NCI-H249 cells treated with tomatidine or cyclopamine (5 μM). Percentages in each phase of the cell cycle are shown below and are shown as the mean of three experiments.

FIG. 3f is a pictoral diagram showing cleaved PARP expression in NCI-H249 and NCI-H417 SCLC cells treated with tomatidine (−) or cyclopamine (+) (5 μM).

FIG. 3g is a pictoral diagram showing Ptch MRNA expression in NCI-H249 SCLC cells detected by northern blot analysis after treatment with cyclopamine. 28s RNA stained with ethidium bromide is shown as a loading control.

FIG. 3h is a pictoral diagram showing RT-PCR analysis of transcripts in NCI-H249 SCLC cells. Cont, control; Tom, tomatidine treated; Cyc, cyclopamine treated.

FIG. 4a is a pictoral diagram showing soft agar growth of NCI-H249 SCLC cells. The top panel shows growth of the cells treated with cyclopamine. Plates were stained with ethidium bromide. The bottom panel shows colony formation of NCI-H249 SCLC cells treated with cyclopamine (5 μM) and stably transfected with neomycin resistance (Neo^{r}), mutant GLI-1 (Flag-Gli1ZFD) or GLI-1 (Flag-Gli1).

FIG. 4b is a graphical representation showing quantitative analysis of the experiment described in 4a. Data is shown as mean colonies per well±s.e.m. (n=6).

FIG. 4c is a graphical representation showing growth of NCI-H249 nude mouse subcutaneous xenografts in animals treated with tomatidine or cyclopamine for 10 days. Data is shown as mean tumor volume±s.e.m. as a percentage of tumor volume at day 0 (n=7).

FIG. 4d is a graphical representation showing data from an identical experiment to that shown in 4c except that A549 NSCLC cells were used. Data is shown as mean tumor volume±s.e.m. as a percentage of tumor volume at day 0 (n=7).

FIG. 5a is a pictoral diagram showing expression of Hh signaling components in human primary lung cancer specimens detected by immunohistochemistry. The photomicrographs show examples of Shh and GLI-1 immunostaining. The SCLC case demonstrates variable co-expression of Shh and GLI-1 in tumor cells. The NSCLC cases demonstrate Shh expression in one case, and lack of expression in the other. Scale bar=50 μm.

FIG. 5b is a graphical representation showing expression of Hh signaling components in cancer cell lines detected by Western blot. RMS13 is a rhabdomyosarcoma line. Growth response to cyclopamine for each cell line tested is shown on the right. Black=strong expression; gray=moderate expression; white=absent expression.

### DETAILED DESCRIPTION OF THE INVENTION

The present invention is based on the identification of elevated hedgehog (Hh) pathway activity within the airway epithelium during differentiation of small-cell lung cancer (SCLC), a highly aggressive and frequently lethal human tumor with primitive neuroendocrine features. This mode of Hh signaling is characterized by the elaboration and reception of the Sonic hedgehog (Shh) signal within the epithelial compartment, and immediately precedes neuroendocrine differentiation.

Sonic hedgehog (Shh), a mammalian hedgehog (Hh) pathway ligand, mediates epithelial-mesenchymal interactions in lung development by signaling to adjacent lung mesenchyme, as indicated by expression of the Hh receptor and pathway target Patched (Ptch) (see Bellusci, et al., Involvement of Sonic hedgehog (Shh) in mouse embryonic lung growth and morphogenesis. Development 124, 53-63 (1997), which is incorporated herein by reference). Loss of Shh function results in severe lung defects associated with failure of branching morphogenesis (Pepicelli, et al., Sonic hedgehog regulates branching morphogenesis in the mammalian lung. Curr. Biol. 8, 1083-1086 (1998); and Litingtung, et al., Sonic hedgehog is essential to foregut development. Nature Genet. 20, 58-61 (1998)). As developmental pathways regulate progenitor cell fates and differentiation in some regenerating mammalian epithelia (Reya, et al., Stem cells, cancer, and cancer stem cells. Nature 414, 105-111 (2001); and Taipale, et al., The Hedgehog and Wnt signaling pathways in cancer. Nature 411, 349-354 (2001)), it became evident that Hh signaling is important in airway epithelial repair.

As disclosed herein, Hedgehog (Hh) pathway activity dramatically increases invasiveness of SCLC cells and promotes changes in expression of genes known to modulate metastasis. SCLC cells displayed elevated levels of Hh pathway activity that were suppressed by the Hh pathway antagonist cyclopamine. Cyclopamine also suppressed cell growth in vitro and caused regression of xenograft tumors in vivo. Hh pathway activity and SCLC cell growth is driven by endogenous expression of Hh ligands, as indicated by the presence of Sonic hedgehog (SHH) transcript, by the pathway-inhibitory and growth-inhibitory activity of a Hh-neutralizing antibody, and by the dramatic growth-stimulatory activity of exogenously added Hh ligand. These results demonstrate that SCLC is characterized by elevated Hh pathway activity that is essential for tumor growth. Accordingly, the present invention provides methods of treating SCLC characterized by elevated Hh pathway activity as compared with a normal cell, as well as methods of determining whether SCLC is amenable to treatment using a Hh pathway antagonist, and methods of identifying agents useful for treating such tumors.

The term “agonist” refers to an agent or analog that binds productively to a receptor and mimics its biological activity. The term “antagonist” refers to an agent that binds to receptors but does not provoke the normal biological response. Thus, an antagonist potentiates or recapitulates, for example, the bioactivity of patched, such as to repress transcription of target genes. The term “hedgehog antagonist” as used herein refers not only to any agent that may act by directly inhibiting the normal function of the hedgehog protein, but also to any agent that inhibits the hedgehog signaling pathway, and thus recapitulates the function of ptc. The term “hedgehog agonist” likewise refers to an agent which antagonizes or blocks the bioactivity of patched, such as to increase transcription of target genes.

The term “antibody” is meant to include intact molecules of polyclonal or monoclonal antibodies, chimeric, single chain, and humanized antibodies, as well as fragments thereof, such as Fab and F(ab′)₂, Fv and SCA fragments which are capable of binding an epitopic determinant. Monoclonal antibodies are made from antigen containing fragments of the protein by methods well known to those skilled in the art (Kohler, et al., Nature, 256:495, 1975). An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

The term “polynucleotide”, “nucleic acid”, “nucleic acid sequence”, or “nucleic acid molecule” refers to a polymeric form of nucleotides at least four bases in length. The nucleotides of the invention can be deoxyribonucleotides, ribonucleotides in which uracil (U) is present in place of thymine (T), or modified forms of either nucleotide. The nucleotides of the invention can be complementary to the deoxynucleotides or to the ribonucleotides

As used herein, reference to the “Hh pathway” means the Hedgehog signal transduction pathway. The Hh pathway is well known (see, e.g., U.S. Pat. No. 6,277,566 B1; U.S. Pat. No. 6,432,970 B2; Lum and Beachy, Science 304:1755-1759, 2004; and Bale and Yu, Hum. Mol. Genet. 10:757-762, 2001, each of which is incorporated herein by reference). Briefly, SHH, IHH and DHH are a family of secreted proteins that act as ligand (Hh ligands) to initiate the Hh pathway, which is involved in morphogenetic development and proliferation of cells in a variety of tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof.

Hh ligands bind to a receptor complex that includes Patched (PTCH; e.g., PTCH-1 in humans) and Smoothened (SMO), which are G-protein coupled receptor-like polypeptides. PTCH is an integral membrane protein with twelve transmembrane domains that acts as an inhibitor of SMO activation. Hh ligand binding to PTCH results in activation of SMO (see, e.g., Taipale et al., Nature 418:892-897, 2002, which is incorporated herein by reference), resulting in transduction of the signal and activation of the GLI family of transcriptional activators (e.g., GLI-1 and GLI-2, which act as transcriptional activators, and GLI-3, which acts as a transcriptional repressor), which are homologs of the Drosophila cubitis interruptis gene. Several kinases also are believed to be involved in the Hh pathway between SMO and the GLI transcription factors, including, for example, protein kinase A, which can inhibit GLI activity. Suppressor of Fused (SUFU) also interacts directly with GLI transcription factors to repress their activity. In addition, various transcriptional targets such as nestin and BMP4 are regulated by Hh pathway activity.

The Hh signaling pathway specifies patterns of cell growth and differentiation in a wide variety of embryonic tissues. Mutational activation of the Hh pathway, whether sporadic or in Gorlin Syndrome, is associated with tumorigenesis in a limited subset of these tissues, predominantly skin, cerebellum, and skeletal muscle (Wechsler-Reya and Scott, The developmental biology of brain tumors. Ann. Rev. Neurosci. 24, 385-428 (2001); Bale and Yu, The hedgehog pathway and basal cell carcinomas. Hum. Mol. Genet. 10, 757-62 (2001)). Known pathway-activating mutations include those that impair the ability of PTCH (the target of Gorlin Syndrome mutations), a transporter-like Hh receptor (Taipale et al., Patched acts catalytically to suppress the activity of Smoothened. Nature 418, 892-7 (2002), to restrain Smoothened (SMO) activation of transcriptional targets via the GLI family of latent transcription factors. Binding of Hh ligand to PTCH is functionally equivalent to genetic loss of PTCH, in that pathway activation by either requires activity of SMO, a seven transmembrane protein that binds to and is inactivated by the pathway antagonist, cyclopamine (Chen et al., Inhibition of Hedgehog signaling by direct binding of cyclopamine to Smoothened. Genes Dev 16, 2743-8 (2002)).

The term “Hh pathway activity” is used herein to refer to the level of Hedgehog pathway signal transduction that is occurring in cells. Hh pathway activity can be determined using methods as disclosed herein or otherwise known in the art (see, e.g., Berman et al., Medulloblastoma growth inhibition by hedgehog pathway blockade. Science 297, 1559-61 (2002); Chen et al., Small molecule modulation of Smoothened activity. Proc Natl Acad Sci USA99, 14071-6 (2002)). As used herein, the term “elevated” or “abnormally elevated”, when used in reference to Hh pathway activity, means that the Hh pathway activity is increased above the level typically found in normal (i.e., not cancer) differentiated cells of the same type as the cancer cells being examined. As such, the term “elevated Hh pathway activity” refers to the level of Hh pathway activity in SCLC cells as compared to corresponding normal cells. Generally, elevated Hh pathway activity is at least about 20% (e.g., 30%, 40%, 50%, 60%, 70%, or more) greater than the Hh pathway activity in corresponding normal cells. In this respect, it should be recognized that Hh pathway activity is determined with respect to a population of cells, which can be a population of cancer cells or a population of normal cells, and, therefore, is an average activity determined from the sampled population.

Reference herein to “normal cells” or “corresponding normal cells” means cells that are from the same organ and of the same type as the cancer cell type. In one aspect, the corresponding normal cells comprise a sample of cells obtained from a healthy individual. Such corresponding normal cells can, but need not be, from an individual that is age-matched and/or of the same sex as the individual providing the cancer cells being examined. In another aspect, the corresponding normal cells comprise a sample of cells obtained from an otherwise healthy portion of tissue of a subject having SCLC.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy (i.e., biopsy sample). In other embodiments, the biological sample of the present invention is a sample of bodily fluid, e.g., serum, plasma, sputum, lung aspirate, urine, and ejaculate.

Accordingly, the invention provides methods of reducing or inhibiting Hh pathway activity and/or proliferation or metastasis of SCLC cells characterized by elevated or abnormally elevated Hh pathway activity. As used herein, the terms “reduce” and “inhibit” are used together because it is recognized that, in some cases, a decrease, for example, in Hh pathway activity can be reduced below the level of detection of a particular assay. As such, it may not always be clear whether the activity is “reduced” below a level of detection of an assay, or is completely “inhibited”. Nevertheless, it will be clearly determinable, following a treatment according to the present methods, that the level of Hh pathway activity (and/or cell proliferation or metastasis) is at least reduced from the level before treatment. Generally, contact of SCLC cells having elevated Hh pathway activity with a Hh pathway antagonist reduces the Hh pathway activity by at least about 20% (e.g., 30%, 40%, 50%, 60%, 70%, or more). For example, the Hh pathway activity in a SCLC cell treated according to the present methods can be reduced to the level of Hh pathway activity typical of a corresponding normal cell.

A Hh pathway antagonist useful in the methods of the invention generally acts at or downstream of the position in the Hh pathway that is associated with the elevated Hh pathway activity. For example, where elevated Hh pathway activity is ligand stimulated, the Hh antagonist can be selected based on the ability, for example, to sequester the Hh ligand (e.g., an antibody specific for the Hh ligand) or to reduce or inhibit binding of the Hh ligand to its receptor. Since Hh ligand activity is dependent on autoprocessing of the Hh ligand (e.g., SHH) into a C-terminal fragment, and an N-terminal fragment that is further modified by attachment of cholesterol and palmitate molecules (and constitutes the ligand; see, e.g., Mann and Beachy, Ann. Rev. Biochem. 73:891-923 (2004), which is incorporated herein by reference), ligand stimulated Hh pathway activity also can be reduced or inhibited by inhibiting autocleavage of the Hh ligand. Where elevated Hh pathway activity is due to an inactivating mutation of the Hh ligand receptor (e.g., PTCH), the Hh pathway antagonist can be selected based on the ability, for example, to sequester SMO (e.g., an antibody specific for SMO) or to reduce activity of a GLI transcription factor (e.g., a polynucleotide comprising a GLI regulatory element, which can act to sequester GLI); an anti-Hh ligand antibody may not necessarily reduce or inhibit elevated Hh pathway activity due to a mutation of PTCH because Hh ligand acts upstream of the defect in the Hh pathway. Further, steroidal alkaloids, such as cyclopamine, its more potent analog KAAD-cyclopamine, and derivatives thereof, and other small molecules such as SANT-1, SANT-2, SANT-3, and SANT-4 can reduce or inhibit elevated Hh pathway activity by directly repressing SMO activity. In addition, cholesterol can be required for Hh pathway activity and, therefore, agents that reduce the availability of cholesterol, for example, by removing it from cell membranes, can act as Hh pathway antagonists (see, e.g., Cooper et al., Nat. Genet 33:508-513 (2003), which is incorporated herein by reference; see, also, Cooper et al., Nat. Genet. 34:113 (2003)).

A Hh pathway antagonist useful in a method of the invention can be any antagonist that interferes with Hh pathway activity, thereby decreasing the elevated or abnormally elevated Hh pathway in SCLC cells. As such, the Hh pathway antagonist can be a peptide, a polynucleotide, a peptidomimetic, a small organic molecule, or any other molecule. Hh pathway antagonists are exemplified by antibodies, including anti-SHH antibodies, anti-IHH antibodies, and/or anti-DHH antibodies, each of which can bind to one or more Hh ligands and decrease ligand stimulated Hh pathway activity. Hh pathway antagonists are further exemplified by SMO antagonists such as steroidal alkaloids and derivatives thereof, including, for example, cyclopamine and jervine (see, e.g., Chen et al., Genes Devel. 16:2743-2748, 2002; and U.S. Pat. No. 6,432,970 B2, each of which is incorporated herein by reference), and SANT-1, SANT-2, SANT-3, and SANT-4 (see Chen et al., Proc. Natl. Acad. Sci., USA 99:14071-14076, 2002, which is incorporated herein by reference); triparanol provides another example of an agent that can act as a Hh pathway antagonist (see, e.g., U.S. Pat. No. 6,432,970 B2). As exemplified herein, an anti-SHH antibody and cyclopamine effectively reduced elevated Hh pathway activity in SCLC cells and reduced viability of the cells in vitro, and cyclopamine suppressed growth of SCLC tumor xenografts in nude mice.

In one aspect, the present invention provides a method of ameliorating SCLC in a subject. As used herein, the term “ameliorate” means that the clinical signs and/or the symptoms associated with SCLC are lessened. The signs or symptoms to be monitored will be characteristic of a particular pulmonary tumor and will be well known to the skilled clinician, as will the methods for monitoring the signs and conditions. For example, the skilled clinician will know that the size or rate of growth of a tumor can be monitored using a diagnostic imaging method typically used for the particular pulmonary tumor (e.g., using ultrasound or magnetic resonance image (MRI) to monitor a pulmonary tumor).

A pulmonary tumor for which Hh pathway activity and cell proliferation or metastasis can be reduced or inhibited can be any tumor of the lung that is characterized, at least in part, by Hh pathway activity that is elevated above levels that are typically found in a normal cell corresponding to the tumor cell. As such, the pulmonary tumor, which can be a benign tumor or can be a malignant tumor, is exemplified herein by small-cell carcinoma or SCLC, mixed cell/large cell carcinoma, and combined small-cell carcinoma (SCLC combined with neoplastic squamous and/or glandular components), each of which is characterized, in part, by elevated or abnormally elevated ligand stimulated Hh pathway activity and increased expression of the Hh ligands SHH and/or IHH.

An agent useful in a method of the invention can be any type of molecule, for example, a polynucleotide, a peptide, a peptidomimetic, peptoids such as vinylogous peptoids, a small organic molecule, or the like, and can act in any of various ways to reduce or inhibit elevated Hh pathway activity when used alone or in combination with cyclopamine. Further, the agent (e.g., a Hh pathway antagonist) can be administered in any way typical of an agent used to treat the particular type of SCLC tumor or under conditions that facilitate contact of the agent with the target tumor cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell.

An agent useful in a method of the invention can be administered to the site of the SCLC tumor, or can be administered by any method that results in the agent contacting the target tumor cells. Generally, the agent is formulated in a composition (e.g., a pharmaceutical composition) suitable for administration to the subject, which can be any vertebrate subject, including a mammalian subject (e.g., a human subject). Such formulated agents are useful as medicaments for treating a subject suffering from SCLC that is characterized, in part, by elevated or abnormally elevated Hh pathway activity.

The term “administration” or “administering” is defined to include an act of providing a compound of the invention or pharmaceutical composition to the subject in need of treatment. The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, inhalation or subcutaneous administration.

The antagonists of the invention may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The agent, which acts as a Hh pathway antagonist to reduce or inhibit the elevated Hh pathway activity, can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1(CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest. 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of a composition containing the Hh pathway antagonist will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the agent is a small organic molecule such as a steroidal alkaloid (e.g., cyclopamine), it can be administered in a form that releases the active agent at the desired position in the body (e.g., the stomach), or by injection into a blood vessel that the agent circulates to the target cells (e.g., SCLC cells).

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

A composition containing a Hh pathway antagonist can be administered to an individual by various routes including, for example, orally or parenterally, such as intravenously, intramuscularly, subcutaneously, intraperitoneally, intrarectally, intracisternally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of a tumor, for example, intravenously or intra-arterially into a blood vessel supplying the tumor.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the Hh pathway antagonist to treat SCLC in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day which can be administered in single or multiple doses.

If desired, the effective daily dose of the active compound may be administered as two, three, four, five, six or more sub-doses administered separately at appropriate intervals throughout the day, optionally, in unit dosage forms. There may be a period of no administration followed by another regimen of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

When other therapeutic agents are employed in combination with the compounds of the present invention they may be used for example in amounts as noted in the Physician Desk Reference (PDR) or as otherwise determined by one having ordinary skill in the art.

The term “effective amount” is defined as the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality. For example, a “therapeutically effective amount” of, e.g., a Hh antagonist, with respect to the subject method of treatment, refers to an amount of the antagonist in a preparation which, when applied as part of a desired dosage regimen brings about, e.g., a change in the rate of cell proliferation and/or the state of differentiation and/or the rate of metastasis of a cell and/or rate of survival of a cell according to clinically acceptable standards for the disorder to be treated.

The term “pharmaceutically acceptable”, when used to identify a carrier, is defined as a carrier, whether diluent or excipient, that is compatible with the other ingredients of the formulation and not deleterious to the recipient thereof. The pharmaceutical composition of the invention can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The invention also provides a method of determining whether SCLC of a subject is amenable to treatment with a Hh pathway antagonist as disclosed herein. The method can be performed, for example, by measuring the level Hh pathway activity in a SCLC cell sample of a subject to be treated, and determining that Hh pathway activity is elevated or abnormally elevated as compared to the level of Hh pathway activity in corresponding normal cells, which can be a sample of normal (i.e., not tumor) cells of the subject having SCLC. Detection of elevated or abnormally elevated level Hh pathway activity in the SCLC cells as compared to the corresponding normal cells indicates that the subject can benefit from treatment with a Hh pathway antagonist. A sample of cells used in the present method can be obtained using a biopsy procedure (e.g., a needle biopsy), or can be a sample of cells obtained by a surgical procedure to remove and/or debulk the tumor.

Elevated or abnormally elevated Hh pathway activity can be determined by measuring elevated expression of one or more (e.g., 1, 2, 3, or more) Hh pathway polypeptide(s), including, for example, one or more Hh ligands (e.g., SHH, IHH, and/or desert hedgehog), Hh ligand receptors (e.g., PTCH), or transcription factors (a GLI family member such as GLI-1), or a combination of such Hh pathway polypeptides. The elevated expression can be detected by measuring the level of a polynucleotide encoding the Hh pathway polypeptide (e.g., RNA) using, for example, a hybridization assay, a primer extension assay, or a polymerase chain reaction (PCR) assay (e.g., a reverse transcription-PCR assay); or by measuring the level the Hh pathway polypeptide(s) using, for example, an immunoassay or receptor binding assay. Alternatively, or in addition, elevated activity of one or more (e.g., 1, 2, 3, or more) Hh pathway polypeptide(s) can be determined. For example, elevated activity of Hh pathway transcription factor (e.g., a GLI family member such as GLI-1) can be detected by measuring increased binding activity of the transcription factor to a cognate transcription factor regulatory element (e.g., using an electrophoretic mobility shift assay), or by measuring increased expression of a reporter gene comprising a cognate transcription factor regulatory element. Expression of a Hh pathway polypeptide having an inactivating mutation can be identified using, for example, an antibody that specifically binds to the mutant, but not to the normal (wild type), Hh polypeptide, wherein the mutation is associated with elevated Hh pathway activity. For example, common mutations that result in expression of an inactivated PTCH can define unique epitopes that can be targeted by diagnostic antibodies that specifically bind the mutant, but not wild type, PTCH protein.

The method of identifying SCLC amenable to treatment with a Hh pathway antagonist can further include contacting cells of the sample with at least one Hh pathway antagonist, and detecting a decrease in Hh pathway activity in the cells following said contact. The decreased Hh pathway activity can be detected, for example, by measuring decreased expression of a reporter gene regulated by a Hh pathway transcription factor, or by detecting a decrease in proliferation or metastasis of the tumor cells. Such a method provides a means to confirm that the SCLC is amenable to treatment with a Hh pathway antagonist. Further, the method can include testing one or more different Hh pathway antagonists, either alone or in combination, thus providing a means to identify one or more Hh pathway antagonists useful for treating the particular SCLC tumor being examined. Accordingly, the present invention also provides a method of identifying an agent useful for treating SCLC having elevated Hh pathway activity.

The method of identifying an agent useful for treating SCLC provides a means for practicing personalized medicine, wherein treatment is tailored to a patient based on the particular characteristics of the SCLC in the subject. The method can be practiced, for example, by contacting a sample of SCLC cells with at least one test agent, wherein a decrease in Hh pathway activity in the presence of the test agent as compared to Hh pathway activity in the absence of the test agent identifies the agent as useful for treating SCLC. The sample of cells examined according to the present method can be obtained from the subject to be treated, or can be cells of an established SCLC cell line of the same type of tumor as that of the subject. In one aspect, the established SCLC cell line can be one of a panel of such cell lines, wherein the panel can include different cell lines of the same type of tumor and/or different cell lines of different tumors. Such a panel of cell lines can be useful, for example, to practice the present method when only a small number of tumor cells can be obtained from the subject to be treated, thus providing a surrogate sample of the subject's tumor, and also can be useful to include as control samples in practicing the present methods.

Once disease is established and a treatment protocol is initiated, the methods of the invention may be repeated on a regular basis to evaluate whether the level of Hh activity in the subject begins to approximate that which is observed in a normal subject. The results obtained from successive assays may be used to show the efficacy of treatment over a period ranging from several days to months. Accordingly, the invention is also directed to methods for monitoring a therapeutic regimen for treating a subject having SCLC. Thus, one skilled in the art will be able to recognize and adjust the therapeutic approach as needed.

The present methods can be practiced using test agents that are known to be effective in treating a cancers having elevated Hh pathway activity (e.g., a steroidal alkaloid such as cyclopamine or jervine; and/or other SMO antagonist such as SANT-1 or SANT-2; and/or an anti-Hh ligand antibody such as an anti-SHH antibody) in order to identify one or more agents that are particularly useful for treating the SCLC being examined, or using test agents that are being examined for effectiveness. In addition, the test agent(s) examined according to the present method can be any type of compound, including, for example, a peptide, a polynucleotide, a peptidomimetic, or a small organic molecule, and can be one or a plurality of similar but different agents such as a combinatorial library of test agents, which can be a randomized or biased library or can be a variegated library based on known effective agent such as the known Hh pathway antagonist, cyclopamine (see, for example, U.S. Pat. No. 5,264,563; and U.S. Pat. No. 5,571,698, each of which is incorporated herein by reference). Methods for preparing a combinatorial library of molecules, which can be tested for Hh pathway antagonist activity, are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., supra, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference; each of which is incorporated herein by reference); an oligosaccharide library (York et al., Carb. Res. 285:99-128, 1996; Liang et al., Science 274:1520-1522, 1996; Ding et al., Adv. Expt. Med. Biol. 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem. 37:1385-1401, 1994; Ecker and Crooke, supra, 1995; each of which is incorporated herein by reference).

The method of identifying an agent useful for treating SCLC can performed by contacting the sample of cells ex vivo, for example, in a culture medium or on a solid support. Alternatively, or in addition, the method can be performed in vivo, for example, by transplanting a SCLC cell sample into a test animal (e.g., a nude mouse), and administering the test agent to the test animal. An advantage of the in vivo assay is that the effectiveness of a test agent can be evaluated in a living animal, thus more closely mimicking the clinical situation. Since in vivo assays generally are more expensive, they can be particularly useful as a secondary screen, following the identification of “lead” agents using an in vitro method.

When practiced as an in vitro assay, the methods can be adapted to a high throughput format, thus allowing the examination of a plurality (i.e., 2, 3, 4, or more) of cell samples and/or test agents, which independently can be the same or different, in parallel. A high throughput format provides numerous advantages, including that test agents can be tested on several samples of cells from a single patient, thus allowing, for example, for the identification of a particularly effective concentration of an agent to be administered to the subject, or for the identification of a particularly effective agent to be administered to the subject. As such, a high throughput format allows for the examination of two, three, four, etc., different test agents, alone or in combination, on the SCLC cells of a subject such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test agent(s); or two or more samples of cells obtained from one subject can be examined with same or different amounts of one or a plurality of test agent. In addition, cell samples, which can be of the same or different subjects, can be examined using combinations of test agents and/or known effective agents. Variations of these exemplified formats also can be used to identifying an agent or combination of agents useful for treating SCLC having elevated Hh pathway activity.

When performed in a high throughput (or ultra-high throughput) format, the method can be performed on a solid support (e.g., a microtiter plate, a silicon wafer, or a glass slide), wherein samples to be contacted with a test agent are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for Hh pathway activity and/or cell viability.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Hedgehog Signaling Within Airway Epithelial Progenitors and in Small-Cell Lung Cancer (SCLC)

The following example demonstrates that small-cell lung cancer (SCLC) cells display elevated Hh pathway activity, and that cyclopamine, a Hh pathway antagonist, can decrease the elevated Hh pathway activity and inhibit proliferation and/or metastasis of the SCLC cells.

Embryonic signaling pathways have been shown to regulate progenitor cell fates in mammalian epithelial development and cancer (Reya, et al., Stem cells, cancer, and cancer stem cells. Nature 414, 105-111 (2001) and Taipale, et al., The Hedgehog and Wnt signaling pathways in cancer. Nature 411, 349-354 (2001)). Prompted by the requirement for sonic hedgehog (Shh) signaling in lung development (Pepicelli, et al., Sonic hedgehog regulates branching morphogenesis in the mammalian lung. Curr. Biol. 8, 1083-1086 (1998) and Litingtung, et al., Sonic hedgehog is essential to foregut development. Nature Genet. 20, 58-61 (1998)), the role of this pathway in regeneration and carcinogenesis of airway epithelium was investigated. It was demonstrated that extensive activation of the hedgehog (Hh) pathway within the airway epithelium during repair of acute airway injury. This mode of Hh signaling is characterized by the elaboration and reception of the Shh signal within the epithelial compartment, and immediately precedes neuroendocrine differentiation. A similar pattern of Hh signaling in airway development was observed during normal differentiation of pulmonary neuroendocrine precursor cells, and in a subset of small-cell lung cancer (SCLC). These tumors maintain their malignant phenotype in vitro and in vivo through ligand-dependent Hh pathway activation. The requirement for Hh pathway activation identifies a common lethal malignancy that may respond to pharmacological blockade of the Hh signaling pathway. Furthermore, cyclopamine inhibition of Hh pathway activity blocks lethality in mice of the highly aggressive SCLC, whereas over-expression of GLI, a transcriptional effector of the Hh pathway, protects SCLC cells from the growth-inhibitory effect of cyclopamine. The role of Hh pathway activity in promoting cellular growth suggests that pathway antagonists may offer significant therapeutic improvements in the treatment of SCLC.

### A. Detection of β-gal Expression

Ptch-LacZ mice were maintained and genotyped as described (Goodrich, et al., Altered neural cell fates and medulloblastoma in mouse patched mutants. Science 277, 1109-1113 (1997)). 5-bromo-4-chloro-3-indolyl-β-D-galactoside (X-gal) staining in microdissected mouse lungs was performed overnight as described (Hogan, et al., Manipulating the Mouse Embryo (Cold Spring Harbor Press, Plainview, 1994), followed by post-fixation in formalin, paraffin embedding and sectioning. Wild-type littermates were used as negative controls.

### B. Immunohistochemistry

Single-color DAB-immunoperoxidase staining was performed using a modification of the DAKO CSA system. Antibodies were from Santa Cruz Biotechnologies: Shh (N-19; sc-1194); GUI (N-16; sc-6153); CGRP (N-20; sc-8856). Shh, Ptch and GLI-1 staining was optimized on paraffin sections from Shh wild-type and knockout embryos. GLI-1 staining was further confirmed in Flag-Gli1-overexpressing Cos-7 cells by immunofluorescence. Peptide competition ablated staining in tumor samples and embryos. Dual-color immunohistochemistry was performed using the DAKO Envision system. Dual-color immunofluorescence was performed on fresh-frozen sections fixed in paraformaldehyde using Molecular Probes Alexa secondary antibodies.

### C. Western Immunoblot

Whole-cell lysates were sonicated in 2% SDS/50 mM TrisHCl, pH 8. Western blot using rabbit polyclonal antibodies for Shh-N were performed as described (Chang, et al., Products, genetic linkage and limb patterning activity of a murine hedgehog gene. Development 120, 3339-3353 (1994)). A rabbit polyclonal antibody to GLI-1 was developed as described (Wang, et al., Hedgehog-regulated processing of GLI-3 produces an anterior/posterior repressor gradient in the developing vertebrate limb. Cell 100, 423-434 (2000)) using a glutathione S-transferase fusion protein containing amino acid residues 216-271 of human GLI-1. Anti-cleaved PARP was obtained from Promega.

### D. RNAse Protection Assay and Northern Blot Analysis

RNAse protection assay (RPA) was performed as described (Sriuranpong, et al., Notch signaling induces rapid degradation of achaete-scute homolog 1. Mol. Cell Biol. 22, 3129-3139 (2002)) using a Ptch-specific antisense RNA probe corresponding to bases 1338-1788 of the human patched-1 cDNA (GI:1335863) generated by RT-PCR and subcloned into pCR-TOPOII (Stratagene). Northern blotting of 10 μg total RNA was performed as described (Nakakura, E. K. et al. Mammalian Scratch: a neural-specific Snail family transcriptional repressor. Proc. Natl. Acad. Sci. USA 98, 4010-4015 (2001)), and probed with a Ptch-specific cDNA probe obtained from the same construct.

### E. RT-PCR

Total cellular RNA was treated with DNAse, reverse transcribed, and amplified for 31 cycles at an annealing temperature of 55° C. Primers used were:

| | |
|---|---|
| BMP4(+): | |
| 5′-CTTTACCGGC TTCAGTCTGGG-3′; | (SEQ ID NO: 1) |
| BMP4(−): | |
| 5′-CCCAATTCCCACTCCCTTGAG-3′; | (SEQ ID NO: 2) |
| GAPDH(+): | |
| 5′-ATCTTCCAGGAGCGAGATCCC-3′; | (SEQ ID NO: 3) |
| GAPDH(−): | |
| 5′-CGTTCGGCTCAGGGATGA CCT-3′; | (SEQ ID NO: 4) |
| ASH-1(+): | |
| 5′-CGCATGGAAAGCTCTGCCAAG-3′; | (SEQ ID NO: 5) |
| ASH-1(−): | |
| 5′-TGACC AACTTGACGCGGTTGC-3′; | (SEQ ID NO: 6) |
| nestin(+): | |
| 5′-CTCTGGGAGAGGAGATTCAAG-3′; | (SEQ ID NO: 7) |
| and | |
| nestin(−): | |
| 5′-CCTTTGTCAGAGGTCTCAGTG-3′. | (SEQ ID NO: 8) |

### F. Shh-LIGHT2 Reporter Assay

Superconfluent reporter cells were cultured as described (Taipale, J. et al., Effects of oncogenic mutations in Smoothened and Patched can be reversed by cyclopamine. Nature 406, 1005-1009 (2000)), then co-cultured in low serum conditions in the presence of 1×10⁵ cells per well of the cell line of interest or purified Shh-Np (Taipale (2000), supra). Luciferase and Renilla luciferase assays were performed using the Promega Dual Luciferase Reporter Assay system.

### G. Cell Culture Experiments

Cell lines were obtained from American Type Culture Collection (ATCC). Shh inhibitor experiments were performed in 0.5% calf serum. Cyclopamine was obtained from Toronto Research Chemicals. Both were dissolved as ×1,000 stocks in DMSO medium. Flag-tagged GLI-1 vectors were obtained from the Joyner laboratory (Park, et al., Mouse GLI-1 mutants are viable but have defects in SHH signaling in combination with a GLI-2 mutation. Development 127, 1593-1605 (2000)). To generate NCI-H249 SCLC cells over expressing each of the GLI vectors, mass cultures were stably co-transfected using lipofectamine (Invitrogen) with the Flag-GLI vector of interest, and pcDNA3.1 (Stratagene) to confer neomycin resistance. 5E1 anti-ShhN monoclonal antibody was used at a concentration of 10 μg ml⁻¹ as described (Ericson, et al., Two critical periods of Sonic Hedgehog signaling required for the specification of motor neuron identity. Cell 87, 661-673 (1996)). Soft agar assays were performed as described (Taipale (2000), supra). Cells were seeded into six-well plates at a density of 20,000 cells per well in agar containing 2% calf serum. MTT assays were performed as described (Sriuranpong (2002), supra).

### H. Nude Mouse Xenografts

Tumor cell lines were injected subcutaneously at 1×10⁷ cells per mouse and allowed to grow to a maximum diameter of 5 mm. Cyclopamine was administered as described (Berman, et al., Medulloblastoma growth inhibition by Hedgehog pathway blockade. Science 297, 1559-1561 (2002)). Tumors were measured daily and the tumor volume calculated as described (Park, et al., Genetic disruption of PPARdelta decreases the tumorigenicity of human colon cancer cells. Proc. Natl. Acad. Sci. USA 98, 2598-2603 (2001)).

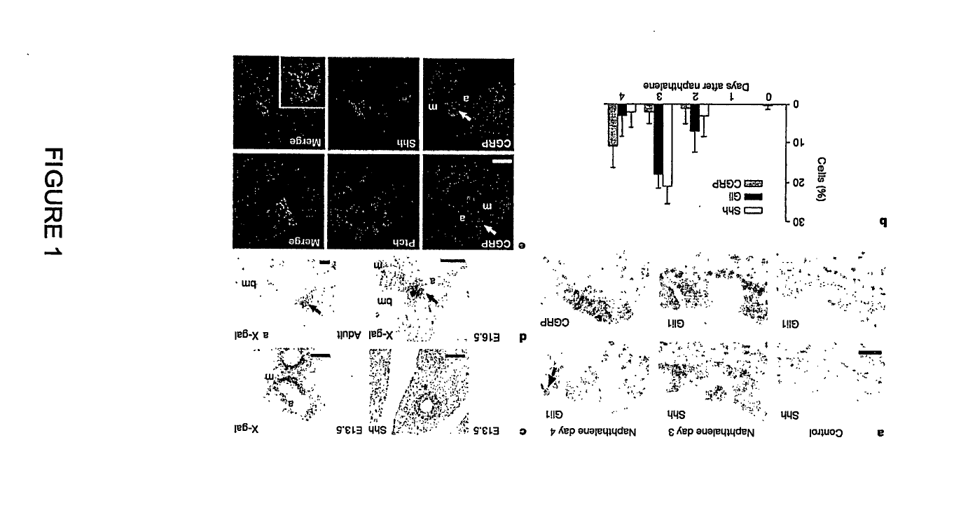

To uncover the role of Hh signaling in this process, a mouse model of acute airway repair, in which Clara cells, specialized airway epithelial cells predominant in distal conducting airways, are depleted within 24 h of systemic naphthalene administration (Reynolds, et al., Neuroepithelial bodies of pulmonary airways serve as a reservoir of progenitor cells capable of epithelial regeneration. Am. J Pathol. 156, 269-278 (2000)) was studied. Activation of a putative airway progenitor results in epithelial regeneration within three days, with increased numbers of airway neuroendocrine cells—a normally rare cell type implicated in the regulation of airway epithelial growth and development (Reynolds (2000), supra; Peake, et al., Alteration of pulmonary neuroendocrine cells during epithelial repair of naphthalene-induced airway injury. Am. J. Pathol. 156, 279-286 (2000)). In regenerating airways, it was observed that marked expression of both Shh ligand and GLI-1, a transcriptional target of Hh signaling (Lee, et al., Gli1 is a target of Sonic hedgehog that induces ventral neural tube development. Development 124, 2537-2552 (1997)), in the epithelial compartment 72 h after naphthalene injury (FIG. 1a). By day 4, GLI-1 was not observed in nascent airway epithelial cells expressing calcitonin gene-related peptide (CGRP), a marker of neuroendocrine differentiation (FIG. 1a, b). The data shows that acute airway epithelial regeneration results in widespread activation of airway intraepithelial Hh signaling, which immediately precedes neuroendocrine differentiation.

Embryonic lung epithelial cells express Shh, which is thought to signal to adjacent lung mesenchyme to regulate branching morphogenesis (Pepicelli, et al., Sonic hedgehog regulates branching morphogenesis in the mammalian lung. Curr. Biol. 8, 1083-1086 (1998); Litingtung, et al., Sonic hedgehog is essential to foregut development. Nature Genet. 20, 58-61 (1998); and Bellusci, et al., Involvement of Sonic hedgehog (Shh) in mouse embryonic lung growth and morphogenesis. Development 124, 53-63 (1997)). In light of this, the detection of Shh and GLI-1 within the epithelial compartment during airway epithelial regeneration was unexpected. To determine whether such intraepithelial signaling occurred in embryonic lung development, mice in which one copy of Ptch is replaced in-frame with the β-galactosidase (β-gal) gene by homologous recombination (Goodrich (1997), supra) were studied. As Ptch is a transcriptional target of the GLI proteins, expression of β-gal indicates activation of the Hh pathway (Goodrich (1997), supra; Taipale (2000), supra). Early gestation (embryonic day (E) 13.5) embryos showed expression of Shh protein in the primitive lung endoderm, and intense β-gal expression in the adjacent mesenchyme (FIG. 1c). By contrast, later lung development (E16.5) was characterized by clusters of β-gal-expressing cells in the developing airway epithelium (FIG. 1d). Small numbers of cells expressing β-gal persist in the basal layer of the adult bronchial epithelium (FIG. 1d). Similar clusters of epithelial cells expressing the neuroendocrine marker CGRP and Ptch were observed by confocal immunofluorescence in E16.5 airways, immediately adjacent to cells expressing Shh (FIG. 1e). This data suggests that during normal development, neuroendocrine precursors within the airway epithelial compartment respond to a Shh signal elaborated by adjacent airway epithelial cells.

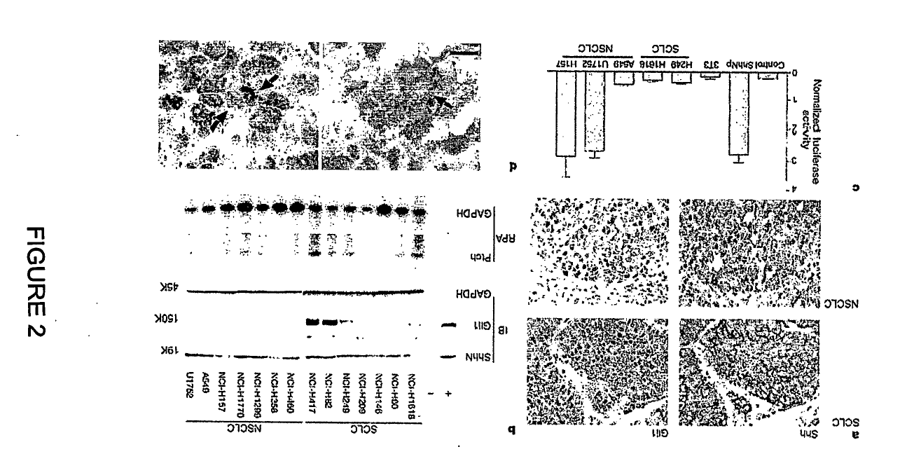

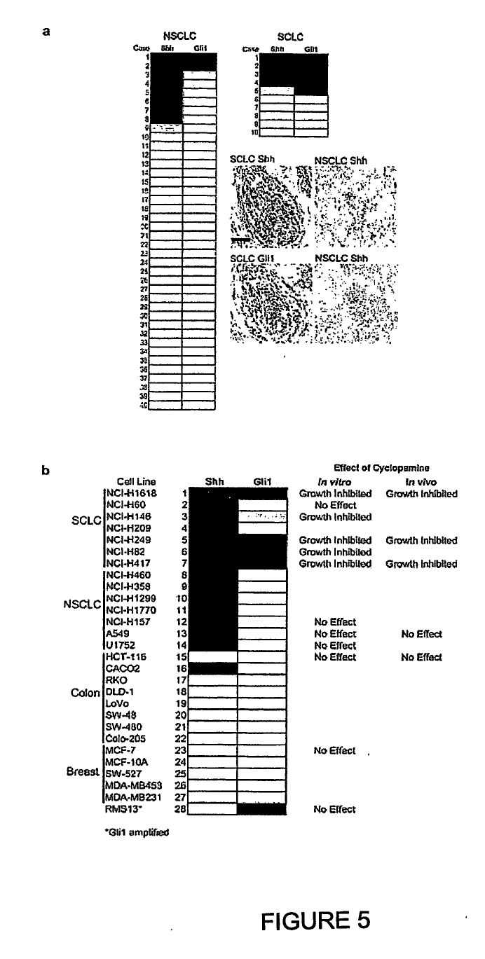

SCLC is an aggressive, highly lethal malignancy with primitive neuroendocrine features (Zochbauer-Muller, et al., Molecular pathogenesis of lung cancer. Annu. Rev. Physiol. 64, 681-708 (2002)). As aberrant reactivation of developmental pathways may have a role in cancer growth (Reya, et al., Stem cells, cancer, and cancer stem cells. Nature 414, 105-111 (2001); and Taipale, et al., The Hedgehog and Wnt signaling pathways in cancer. Nature 411, 349-354 (2001)), the determination as to whether the epithelial Hh signaling that was observed in airway embryogenesis and repair persisted in SCLC. Analysis of SCLC tissue showed that five out of ten tumors expressed both Shh and GLI-1 (FIG. 2a; see also FIG. 5a). Out of 40 non-SCLC (NSCLC) tumors, nine demonstrated Shh expression and of these, four demonstrated co-expression of GLI-1 (FIG. 2a; see also FIG. 5a). The data provides indirect evidence of persistent activation of Hh signaling in lung cancer, predominantly in SCLC. These findings were confirmed by analysis of human lung cancer cell lines. Notably, all seven SCLC and seven NSCLC cell lines expressed Shh protein (FIG. 2b). Out of five breast and eight colon cancer cell lines examined, only one (CACO2) expressed Shh protein, and none expressed GLI-1 protein, as shown by western blot analysis (data not shown). Importantly, expression of both Shh and GLI-1 proteins was observed in five out of seven SCLC lines, and this correlated with increased expression of Ptch messenger RNA (FIG. 2b). In contrast, NSCLC lines expressed Shh and low levels of Ptch, but not GLI-1. The data is summarized in FIG. 5b.

To determine how Hh signaling might function in these tumors, cancer cells were co-cultured with Shh-LIGHT2 cells, a fibroblast reporter cell line that responds to exogenous Shh by activation of an integrated GLI-responsive luciferase reporter (Taipale (2000), supra). Some NSCLC cells that express Shh are capable of heterologous cell signaling to the reporter cell line (FIG. 2c), suggesting that NSCLC retains the Shh export properties of primitive lung endo-dermal cells that signal to adjacent mesenchymal cells in early development. By contrast, the SCLC cells examined here display a marked reduction in this ability to signal to adjacent cells. The data demonstrates that distinct types of lung cancer cells recapitulate different aspects of Shh signaling seen in lung development and repair.

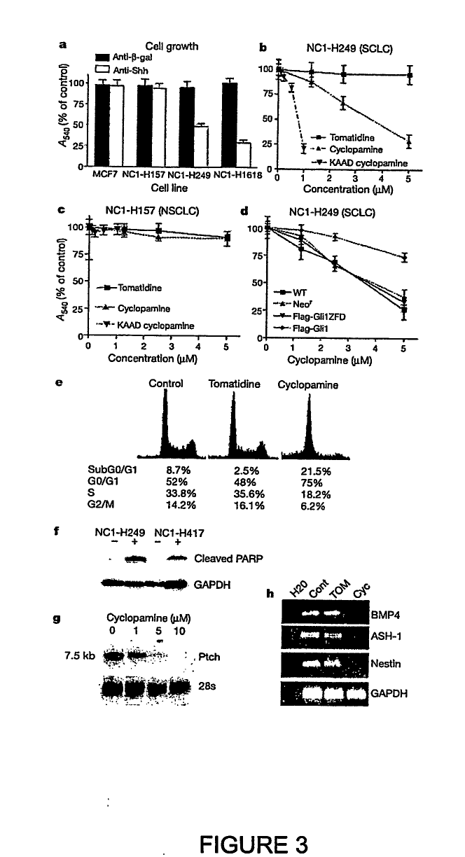

The mechanism of Hh pathway activation in SCLC was then addressed. Dual-label immunostaining for Shh and GLI-1 in SCLC nude mouse xenografts demonstrated Shh-expressing cells adjacent to GLI-1-expressing cells (FIG. 2d). This data suggests juxtacrine Hh pathway activation in SCLC markedly similar to that observed in airway development and repair. Next, it was determined whether ligand-driven Hh pathway activation promotes growth of SCLC. Inhibition of Shh ligand activity in NCI-H249 and NCI-H1618 SCLC cells with the 5E1 Shh-N monoclonal antibody (Ericson (1996), supra) resulted in growth inhibition (FIG. 3a). Although NCI-H157 NSCLC cells express Shh, they do not express GLI-1 protein, and are not affected by 5E1 treatment (FIG. 3a). The data demonstrates that growth of SCLC cells in vitro is dependent on ligand-mediated activation of the Hh pathway, and suggest the presence of a normal Ptch receptor, confirmed by sequencing of Ptch complementary DNA in both NCI-H249 and NCI-H1618 SCLC cells generated by reverse transcription-polymerase chain reaction (RT-PCR) (data not shown).

The Veratrum alkaloid cyclopamine specifically inhibits the Hh pathway (Taipale (2000), supra; Incardona, et al., The teratogenic Veratrum alkaloid cyclopamine inhibits sonic hedgehog signal transduction. Development 125, 3553-3562 (1998); and Cooper, et al., Teratogen-mediated inhibition of target tissue response to Shh signaling. Science 280, 1603-1607 (1998)) through interaction with the Hh signaling protein smoothened (Chen, et al., Inhibition of Hedgehog signaling by direct binding of cyclopamine to Smoothened. Genes Dev. 16, 2743-2748 (2002); and Chen, et al., Small molecule modulation of Smoothened activity. Proc. Natl. Acad. Sci. USA 99, 14071-14076 (2002)). Moreover, cyclopamine blocks the oncogenic effects of mutations of Ptch in fibroblasts (Taipale (2000), supra), and inhibits the malignant growth of medulloblastoma cells lacking Ptch function (Berman (2002), supra). Treatment of NCI-H249 SCLC cells with cyclopamine, or a more potent analogue KAAD-cyclopamine (Taipale (2000), supra), resulted in significant growth inhibition, whereas tomatidine, a closely related compound that lacks the capacity to inhibit Hh signaling, had no effect (FIG. 3b). The effects of cyclopamine and KAAD-cyclopamine on the growth of SCLC reflect their relative potency in silencing Hh pathway activation in vitro (Taipale (2000), supra). None of KAAD-cyclopamine, cyclopamine or tomatidine was able to affect growth of NCI-H157 NSCLC cells (FIG. 3c). The growth-inhibitory effect of cyclopamine, if due to Hh pathway blockade, should be bypassed by constitutive overexpression of the Hedgehog pathway effector GLI-1 (Berman (2002), supra). It was observed that stable expression of a Flag-tagged GLI-1 protein (Park (2000), supra) protected NCI-H249 SCLC cells from growth inhibition by cyclopamine, whereas a GLI-1 mutant lacking the zinc finger DNA-binding domain had no effect (FIG. 3d). Treatment of nine cancer cell lines with cyclopamine at concentrations up to 10 μM demonstrated growth inhibition only in SCLC cells that expressed both Shh and its transcriptional effector GLI-1 (FIG. 5b). This data shows that cyclopamine induces growth inhibition in SCLC cells expressing both Shh and GLI-1 by specific inhibition of the Hh pathway.

The relationship between Hh pathway blockade by cyclopamine and growth arrest in SCLC was then investigated. Unsynchronized NCI-H249 SCLC cells treated with 5 μM cyclopamine for 72 h demonstrated arrest of the cell cycle in Go/G1 (FIG. 3e) and apoptosis indicated by an increase in cleaved PARP (FIG. 3f). Analysis of Ptch mRNA expression revealed downregulation in response to cyclopamine treatment (FIG. 3g). These results indicate silencing of Hh pathway activation at concentrations of cyclopamine that induce both growth arrest and apoptosis. It was then investigated whether SCLC cells might express transcripts indicative of a progenitor cell phenotype. Expression of BMP4, a morphogen and putative target of Hh expressed in lung epithelial embryogenesis (Weaver, et al., Bmp signaling regulates proximal-distal differentiation of endoderm in mouse lung development. Development 126, 4005-4015 (1999)), and nestin, an intermediate filament characteristic of neural stem cells in medulloblastoma (Berman (2002), supra) (FIG. 3h), was detected. Treatment of NCI-H249 SCLC cells with cyclopamine for 48 h inhibited expression of both these genes (FIG. 3h), as well as the expression of human ASH-1, a transcription factor required for pulmonary neuroendocrine differentiation (Borges, et al., An achaete-scute homologue essential for neuroendocrine differentiation in the lung. Nature 386, 852-855 (1997)). These changes in gene expression suggest that Hh signaling maintains a progenitor cell fate in SCLC.

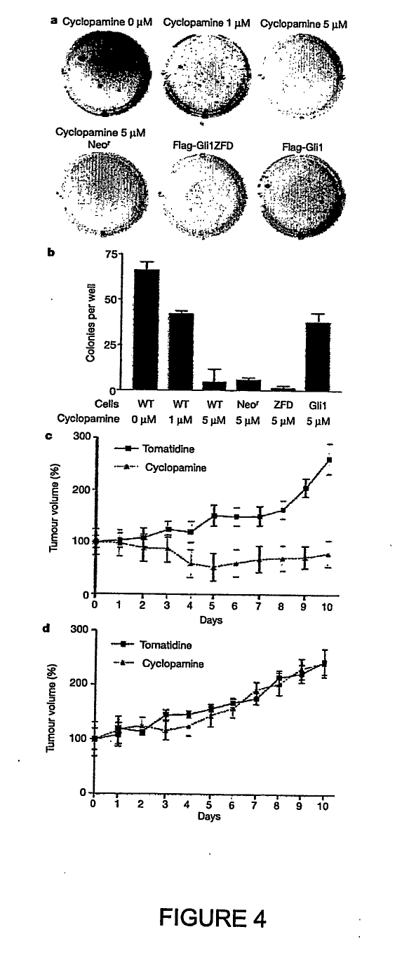

Pathological activation of Hh signaling is associated with medulloblastoma, a malignant brain tumor thought to arise from the granule cells of the cerebellum (Goodrich (1997), supra); and Kenney, et al., Sonic hedgehog promotes G(1) cyclin expression and sustained cell cycle progression in mammalian neuronal precursors. Mol. Cell Biol. 20, 9055-9067 (2000)). Maintenance of abnormal progenitor-like fates through continued Hh pathway activation is essential for malignant growth of these tumors in vivo (Berman (2002), supra). Consequently, it was then determined whether SCLC cells were similarly dependent on Hh signaling for their malignant behavior. NCI-H249 SCLC cells treated with cyclopamine showed reduced soft agar clonogenicity—an in vitro assay of tumorigenicity (FIG. 4a, b). This effect was reversed in cells overexpressing the Hh pathway transcriptional effector GLI-1 (FIG. 4a, b). The ability of systemic cyclopamine treatment to inhibit the growth of SCLC xenografts in nude mice was then determined. Mice bearing xenografts were treated subcutaneously with 25 mg⁻¹ kg⁻day⁻¹ cyclcopamine as described (Berman (2002), supra). Growth inhibition was observed in three SCLC lines: NCI-H249 (FIG. 4c), as well as NCI-H417 and NCI-H1618 (data not shown). No effect was observed in A549 NSCLC cells (FIG. 4d) nor in HCT-116 colon cancer xenografts (data not shown). The data is summarized in FIG. 5b, and shows that Hh signaling is required for the growth in vivo of SCLC cells that express both Shh and GLI-1.

It was shown that Hh signaling in airway epithelium is not limited to epithelial-mesenchymal interactions, but can be contained within the airway epithelial compartment during embryonic neuroendocrine differentiation and airway repair. Taking evidence that links Hh signaling to cerebellar progenitor cell differentiation into consideration (Kenny (2000), supra; Dahmane, et al., Sonic hedgehog regulates the growth and patterning of the cerebellum. Development 126, 3089-3100 (1999); and Wechsler-Reya, et al., Control of neuronal precursor proliferation in the cerebellum by Sonic Hedgehog. Neuron 22, 103-114 (1999)), it was hypothesized that a similar role for this pathway in the regulation of airway progenitor cell fates, which may be specified immediately before the divergence of neuroendocrine and non-neuroendocrine lineages. The dependency of SCLC cells on Hh pathway activation is also notable in that it relies on the presence of Shh ligand, it occurs in the absence of mutations in Ptch, and recapitulates juxtacrine Hh signaling seen in development and airway repair. SCLC may represent a malignancy arising from an airway epithelial progenitor that retains both Hh signaling and primitive features of pulmonary neuroendocrine differentiation. The vulnerability of SCLC to Hh pathway blockade may represent a new therapeutic approach to a disease with a poor prognosis (Johnson, D. H., Management of small cell lung cancer*: current state of the art. Chest 116,525S-530S (1999)).

Although the invention has been described with reference to the above examples, it will be understood that modifications and variations are encompassed within the spirit and scope of the invention. Accordingly, the invention is limited only by the following claims.

What is claimed is:

1. A method of reducing or inhibiting proliferation or metastasis of small-cell lung cancer (SCLC) cells characterized by elevated Hedgehog (Hh) pathway activity as compared with a normal cell, comprising contacting the cells with at least one Hh pathway antagonist, thereby reducing or inhibiting proliferation of the small-cell lung cancer cells.

2. The method of claim 1, wherein the elevated Hh pathway activity comprises elevated ligand stimulated Hh pathway activity.

3. The method of claim 2, wherein the ligand comprises Sonic hedgehog (SHH).

4. The method of claim 1, wherein the elevated Hh pathway activity comprises elevated transcription factor.

5. The method of claim 4, wherein the transcription factor comprises a GLI-1 transcription factor.

6. The method of claim 1, wherein the Hh pathway antagonist comprises a steroidal alkaloid or derivative thereof.

7. The method of claim 6, wherein the steroidal alkaloid is cyclopamine.

8. The method of claim 1, wherein the Hh pathway antagonist is a nucleic acid or a protein molecule.

9. The method of claim 8, wherein the protein molecule is an antibody or binding fragment thereof.

10. The method of claim 1, further comprising contacting the cells with a chemotherapeutic agent.

11. The method of claim 7, further comprising contacting the cells with an antibody or binding fragment thereof.

12. The method of claim 11, wherein the antibody is an anti-Hh antibody.

13. A method of ameliorating small-cell lung cancer in a subject, comprising administering to the subject a Hh pathway antagonist, whereby the Hh pathway antagonist contacts small-cell lung cancer cells in the subject, thereby ameliorating the small-cell lung cancer in the subject.

14. The method of claim 13, wherein the elevated Hh pathway activity comprises elevated ligand stimulated Hh pathway activity.

15. The method of claim 14, wherein the ligand comprises Sonic hedgehog (SHH).

16. The method of claim 13, wherein the elevated Hh pathway activity comprises elevated transcription factor.

17. The method of claim 16, wherein the transcription factor comprises a GLI-1 transcription factor.

18. The method of claim 13, wherein the Hh pathway antagonist comprises a steroidal alkaloid or derivative thereof.

19. The method of claim 18, wherein the steroidal alkaloid is cyclopamine.

20. The method of claim 13, further comprising administering to the subject a chemotherapeutic agent.

21. The method of claim 19, further comprising contacting the cells with an antibody or binding fragment thereof.

22. The method of claim 21, wherein the antibody is anti-Hh antibody.

23. The method of claim 13, wherein the Hh pathway antagonist is administered orally.

24. A method of identifying small-cell lung cancer amenable to treatment with a Hedgehog (Hh) pathway antagonist, comprising detecting elevated Hh pathway activity in a sample of cells as compared to Hh pathway activity in corresponding normal cells, thereby identifying small-cell lung cancer amenable to treatment with a Hh pathway antagonist.

25. The method of claim 24, wherein the cells are from a biopsy sample obtained from a subject.

26. The method of claim 24, wherein the cells are from a tissue or bodily fluid obtained from a subject.

27. The method of claim 24, wherein the elevated Hh pathway activity comprises ligand stimulated Hh pathway activity.

28. The method of claim 24, comprising detecting elevated expression of at least one Hh pathway polypeptide.

29. The method of claim 28, wherein the Hh pathway polypeptide comprises a Hh ligand, a Hh ligand receptor, or a transcription factor.

30. The method of claim 29, wherein the Hh ligand comprises Sonic hedgehog (SHH).

31. The method of claim 29, wherein the Hh ligand receptor comprises Patched.

32. The method of claim 29, wherein the transcription factor comprises a GLI-1 transcription factor.

33. The method of claim 28, which comprises detecting elevated levels of a polynucleotide encoding the Hh pathway polypeptide.

34. The method of claim 33, wherein the polynucleotide comprises RNA.

35. The method of claim 28, which comprises performing a reverse transcription-polymerase chain reaction.

36. The method of claim 28, which comprises detecting elevated levels of the Hh pathway polypeptide.

37. The method of claim 36, which comprises performing an immunoassay.

38. The method of claim 36, wherein the Hh pathway polypeptide comprises a transcription factor.

39. The method of claim 38, which comprises detecting increased binding activity of the transcription factor to a cognate transcription factor regulatory element.

40. The method of claim 38, which comprises detecting increased expression of a reporter gene comprising a cognate transcription factor regulatory element.

41. The method of claim 24, which comprises detecting altered expression of a transcriptional target of the Hh pathway.

42. The method of claim 41, which comprises detecting increased expression of a gene that is positively regulated by GLI-1.

43. The method of claim 24, further comprising contacting cells of the sample with at least one Hh pathway antagonist, and detecting a decrease in Hh pathway activity in the cells following said contact, thereby confirming that the small-cell lung cancer is amenable to treatment with a Hh pathway antagonist.

44. The method of claim 43, wherein the antagonist is cyclopamine.

45. The method of claim 44, further comprising contacting the cells with a chemotherapeutic agent.

46. The method of claim 44, further comprising contacting the cells with an anti-Hh antibody or binding fragment thereof.

47. A method of identifying an agent useful for treating small-cell lung cancer wherein the small-cell lung cancer cells have elevated Hedgehog (Hh) pathway activity, comprising contacting a sample of small-cell lung cancer cells with at least one test agent, wherein a decrease in Hh pathway activity in the presence of the test agent as compared to Hh pathway activity in the absence of the test agent identifies the agent as useful for treating small-cell lung cancer.

48. The method of claim 47, wherein the elevated Hh pathway activity comprises elevated ligand stimulated Hh pathway activity.

49. The method of claim 48, wherein the ligand comprises Sonic hedgehog (SHH).

50. The method of claim 47, wherein the elevated Hh pathway activity comprises elevated transcription factor.

51. The method of claim 50, wherein the transcription factor comprises a GLI-1 transcription factor.

52. The method of claim 47, wherein the agent comprises a Hh pathway antagonist.

53. The method of claim 52, wherein the antagonist comprises steroidal alkaloid or a derivative thereof.

54. The method of claim 49, wherein the steroidal alkaloid is cyclopamine.

55. The method of claim 47, which is performed in a high throughput format.

56. The method of claim 55, comprising contacting samples of cells of a plurality of samples with at least one test agent.

57. The method of claim 56, wherein plurality of samples are obtained from a single subject.

58. The method of claim 56, wherein the plurality of samples are obtained from different subjects.

59. A method for monitoring a therapeutic regimen for treating a subject having small-cell lung cancer comprising determining a change in Hh pathway activity during therapy.

60. The method of claim 59, wherein the therapy comprises the treatment of claim 1.


SEQUENCE LISTING (8 sequences)

SEQ ID NO: 1 - DNA, 21 bp, Artificial sequence
  Amplification primer
ctttaccggcttcagtctggg

SEQ ID NO: 2 - DNA, 21 bp, Artificial sequence
  Amplification primer
cccaattcccactcccttgag

SEQ ID NO: 3 - DNA, 21 bp, Artificial sequence
  Amplification primer
atcttccaggagcgagatccc

SEQ ID NO: 4 - DNA, 21 bp, Artificial sequence
  Amplification primer
cgttcggctcagggatgacct

SEQ ID NO: 5 - DNA, 21 bp, Artificial sequence
  Amplification primer
cgcatggaaagctctgccaag

SEQ ID NO: 6 - DNA, 21 bp, Artificial sequence
  Amplification primer
tgaccaacttgacgcggttgc

SEQ ID NO: 7 - DNA, 21 bp, Artificial sequence
  Amplification primer
ctctgggagaggagattcaag

SEQ ID NO: 8 - DNA, 21 bp, Artificial sequence
  Amplification primer
cctttgtcagaggtctcagtg